SEQUENCE LISTING (20 sequences)

SEQ ID NO: 1 - DNA, 1331 bp, Artemisia annua
gtcttcaacacatcacactttaccactcatcactgatctgatcctggtcacatcaaactcatatgtctgaaaaaccaaccttgttttctgcctacaagatgggcaagttcaatctttctcacagggtggtgttagctcctatgactcggtgtcgggcaataaacgctataccgaacgaagcgcttgtcgaatattatcggcaaagatcaactgctggtggctttcttattaccgaggggactatgatctctccttcttctgccgggtttccgcacgtaccgggtatttttactaaggaacaagtcgagggttggaaaaaagtggttgatgcagctcacaaagaaggggctgtgatattttgtcaattgtggcatgttggtagagcatcccatcaagtgtaccaacccggtggtgctgcgccaatatcatcaacaagcaagcccatttcaaagaagtgggaaatattattgccagatgcaacctatggcacgtatcctgagccccgtccattggcagccaatgaaatactagaggtggttgaagactatcgcgttgcagctattaatgctattgaagcaggttttgatggcattgaaatacatggagcacatggttatctacttgaccagttcatgaaagatggcataaatgacagaacggacgaatatggtgggtcattagaaaaccgctgcaaattcatactgcaagtagtgcaagcggtatctgcagccattggtacagaccgagttggtattagaatctcccctgcaatcgatcacacagatgctatggactctgatccacgaagcctaggccttgcagtaattgaaagactcaacaaactacaatttaaattagggtcaagactagcttatcttcatgtaactcaaccacgttacacggctgatggtcatggtcaaacagaagcgggagccaatggaagtgaggaggaagtagctcagttgatgaaaacatggagaggagcttatgtgggaacttttatatgctgcggtggttacactagagaacttgggttacaagctgtggctcaaggggatgctgatttggtcgcttttggtcgttattttgtctcgaatcctgaccttgttttaagacttaagctcaacgcacctttaaataggtatgacagagctactttttatacccatgatcctgtcgttgggtacacggattacccttctcttgacaaagggtcactcctctagttttgattatattgattgtcaaataacttatgctctattcttattaaggtgcatcttatgagcaaaataaacctgttttcgaaaaaaaaaaaaaaaaaaaaaaaa

SEQ ID NO: 2 - protein, 387 aa, Artemisia annua
MSEKPTLFSAYKMGKFNLSHRVVLAPMTRCRAINAIPNEALVEYYRQRSTAGGFLITEGTMISPSSAGFPHVPGIFTKEQVEGWKKVVDAAHKEGAVIFCQLWHVGRASHQVYQPGGAAPISSTSKPISKKWEILLPDATYGTYPEPRPLAANEILEVVEDYRVAAINAIEAGFDGIEIHGAHGYLLDQFMKDGINDRTDEYGGSLENRCKFILQVVQAVSAAIGTDRVGIRISPAIDHTDAMDSDPRSLGLAVIERLNKLQFKLGSRLAYLHVTQPRYTADGHGQTEAGANGSEEEVAQLMKTWRGAYVGTFICCGGYTRELGLQAVAQGDADLVAFGRYFVSNPDLVLRLKLNAPLNRYDRATFYTHDPVVGYTDYPSLDKGSLL

SEQ ID NO: 3 - DNA, 1248 bp, Artemisia annua
atgtcgtactaccatcaccatcaccatcacctcgaatcaacaagtttgtacaaaaaagcaggctccgcggccgcccccttcaccatgtctgaaaaaccaaccttgttttctgcctacaagatgggcaagttcaatctttctcacagggtggtgttagctcctatgactcggtgtcgggcaataaacgctataccgaacgaagcgcttgtcgaatattatcggcaaagatcaactgctggtggctttcttattaccgaggggactatgatctctccttcttctgccgggtttccgcacgtaccgggtatttttactaaggaacaagtcgagggttggaaaaaagtggttgatgcagctcacaaagaaggggctgtgatattttgtcaattgtggcatgttggtagagcatcccatcaagtgtaccaacccggtggtgctgcgccaatatcatcaacaagcaagcccatttcaaagaagtgggaaatattattgccagatgcaacctatggcacgtatcctgagccccgtccattggcagccaatgaaatactagaggtggttgaagactatcgcgttgcagctattaatgctattgaagcaggttttgatggcattgaaatacatggagcacatggttatctacttgaccagttcatgaaagatggcataaatgacagaacggacgaatatggtgggtcattagaaaaccgctgcaaattcatactgcaagtagtgcaagcggtatctgcagccattggtacagaccgagttggtattagaatctcccctgcaatcgatcacacagatgctatggactctgatccacgaagcctaggccttgcagtaattgaaagactcaacaaactacaatttaaattagggtcaagactagcttatcttcatgtaactcaaccacgttacacggctgatggtcatggtcaaacagaagcgggagccaatggaagtgaggaggaagtagctcagttgatgaaaacatggagaggagcttatgtgggaacttttatatgctgcggtggttacactagagaacttgggttacaagctgtggctcaaggggatgctgatttggtcgcttttggtcgttattttgtctcgaatcctgaccttgttttaagacttaagctcaacgcacctttaaataggtatgacagagctactttttatacccatgatcctgtcgttgggtacacggattacccttctcttgacaaagggtcactcctctag

SEQ ID NO: 4 - protein, 415 aa, Artemisia annua
MSYYHHHHHHLESTSLYKKAGSAAAPFTMSEKPTLFSAYKMGKFNLSHRVVLAPMTRCRAINAIPNEALVEYYRQRSTAGGFLITEGTMISPSSAGFPHVPGIFTKEQVEGWKKVVDAAHKEGAVIFCQLWHVGRASHQVYQPGGAAPISSTSKPISKKWEILLPDATYGTYPEPRPLAANEILEVVEDYRVAAINAIEAGFDGIEIHGAHGYLLDQFMKDGINDRTDEYGGSLENRCKFILQVVQAVSAAIGTDRVGIRISPAIDHTDAMDSDPRSLGLAVIERLNKLQFKLGSRLAYLHVTQPRYTADGHGQTEAGANGSEEEVAQLMKTWRGAYVGTFICCGGYTRELGLQAVAQGDADLVAFGRYFVSNPDLVLRLKLNAPLNRYDRATFYTHDPVVGYTDYPSLDKGSLL

SEQ ID NO: 5 - DNA, 24 bp, Artificial
  Primer
gctcataagatgcaccttaataag

SEQ ID NO: 6 - DNA, 23 bp, Artificial
  Primer
aagcagtggtatcaacgcagagt

SEQ ID NO: 7 - DNA, 25 bp, Artificial
  Primer
caccatgtctgaaaaaccaaccttg

SEQ ID NO: 8 - DNA, 24 bp, Artificial
  Primer
gctcataagatgcaccttaataag

SEQ ID NO: 9 - DNA, 35 bp, Artificial
  Primer
taagcggccgcatgagtagcatcgatctgaaatcc

SEQ ID NO: 10 - DNA, 32 bp, Artificial
  Primer
taaactagtctacttttgcctcttgtagattt

SEQ ID NO: 11 - DNA, 33 bp, Artificial
  Primer
taaggatccatggaacagcaacaagaagtgatc

SEQ ID NO: 12 - DNA, 32 bp, Artificial
  Primer
taagggccctctatactcataggataaacgag

SEQ ID NO: 13 - DNA, 33 bp, Artificial
  Primer
attggatccatgaagagtatactaaaagcaatg

SEQ ID NO: 14 - DNA, 34 bp, Artificial
  Primer
taagtcgacctagaaacttggaacgagtaacaac

SEQ ID NO: 15 - DNA, 35 bp, Artificial
  Primer
attgcggccgcatgcaatcaacaacttccgttaag

SEQ ID NO: 16 - DNA, 34 bp, Artificial
  Primer
tgattaattaattaccatacatcacggagatatc

SEQ ID NO: 17 - protein, 11 aa, Artemisia annua
RSLGLAVIERL

SEQ ID NO: 18 - protein, 12 aa, Artemisia annua
KLAYLHVTQPRY

SEQ ID NO: 19 - protein, 13 aa, Artemisia annua
RTDEYGGSLENRC

SEQ ID NO: 20 - protein, 21 aa, Artemisia annua
RELGLQAVAQGDADLVAFGRH

(19) United States
(12) Patent Application Publication
(10) Pub. No.: US 2010/0299778 A1
(43) Pub. Date: Nov. 25, 2010

(54) NUCLEOTIDE SEQUENCE ENCODING ARTEMISINIC ALDEHYDE DOUBLE BOND REDUCTASE, ARTEMISINIC ALDEHYDE DOUBLE BOND REDUCTASE AND USES THEREOF

(75) Inventors: Yansheng Zhang, Saskatchewan (CA); Thomas Keat Teoh, Jonesboro, AR (US); Darwin W. Reed, Saskatchewan, CA (US); Douglas J.H. Olson, Saskatchewan, CA (US); Andrew R. S. Ross, Victoria (CA); Patrick S. Covello, Saskatoon (CA)

Correspondence Address: NATIONAL RESEARCH COUNCIL OF CANADA, 1200 Montreal Road, Building M-58 Room EG-12, OTTAWA, ON, K1A 0R6, CA

(73) Assignee: National Research Council of Canada

(21) Appl. No.: 12/734,791

(22) PCT Filed: Nov. 19, 2008
(86) PCT No.: PCT/CA2008/002029

§ 371 Date: May 24, 2010

Related U.S. Application Data
(60) Provisional application No. 61/004,564, filed on Nov. 28, 2007.

(51) Int. Cl. A01H 1/04; C07H 21/04; C12N 9/02; C12P 7/24; C12P 7/40; C12P 17/18

(52) U.S. Cl. 800/267; 536/23.2; 435/189; 435/147; 435/136; 435/119

(57) ABSTRACT

An isolated nucleic acid molecule cloned from Artemisia annua encodes an artemisinic aldehyde double bond reductase. Artemisinic aldehyde double bond reductase enzymatically reduces artemisinic aldehyde to (11R)-dihydroartemisinic aldehyde. The nucleic acid molecule, and the enzyme encoded thereby, may be used in processes to produce dihydroartemsinic aldehyde and/or dihydroartemisinic acid in a host cell. Dihydroartemisinic acid is a late precursor to the antimalarial compound artemisinin.

### CROSS-REFERENCE TO RELATED APPLICATIONS

This application claims the benefit of United States Provisional patent application U.S. Ser. No. 61/004,564 filed Nov. 28, 2007, the entire contents of which his herein incorporated by reference.

### FIELD OF THE INVENTION

The present invention relates to production of plant-derived compounds of health and commercial interest. More particularly, the present invention relates to nucleotide sequences encoding enzymes, to enzymes encoded by the nucleotide sequences and to processes for producing dihydroartemisinic aldehyde, dihydroartemisinic acid and/or artemisinin therewith.

### BACKGROUND OF THE INVENTION

Plants, in general, contain a myriad of secondary metabolites often synthesized by unique biochemical processes operating only in exotic species. For plant-derived products such as drugs, the 1997 worldwide sales were US$ 10 billion (Rotheim 2002). In many cases the supply of the relevant plant material for these drugs is limited or variable. One approach to developing methods for producing these drugs is to apply the methods of biochemistry, molecular biology and genomics to elucidate the biosynthesis and relevant biosynthetic genes for compounds of value for human health.

With the realization that many of the enzymes involved in natural product biosynthesis represent variations within known classes of enzymes, expressed sequence tag (EST) analysis (combined with heterologous expression) provides a powerful means of identifying their corresponding genes (Cahoon et al. 1999; Gang et al. 2001; Lange et al. 2000; van de Loo, Turner, & Somerville 1995).

One area of interest is bioactive compounds of the tribe Anthemideae in the family Asteraceae (Compositae) (Torrell et al. 1999; Watson, Evans, & Boluarte 2000). Anthemideae (Asteraceae, subfamily Asteroideae) is a tribe of 109 genera which includes daisies, chrysanthemums, tarragon, chamomile, yarrow and sagebrushes (Watson, Evans, & Boluarte 2000). These plants are aromatic in nature resulting from high concentrations of mono- and sesqui-terpenes. Many of the species in this tribe are valued for the health benefits or insecticidal properties.

Of particular interest is artemisinin from Artemisia annua or sweet wormwood. In 1972, Chinese scientists reported the isolation of the sesquiterpene lactone containing an endoperoxide group (see FIG. 1) from Artemisia and called it qinghaosu (van Agtmael, Eggelte, & van Boxtel 1999). Prior to this sweet wormwood or qinghao had been used in traditional Chinese medicine for centuries. Artemisinin has become very important for the treatment of malaria in Southeast Asia and elsewhere, particularly for multi-drug-resistant falciparum forms of the disease (O'Neill 2005; Rathore et al. 2005; Robert, Coppel, & Meunier 2002; Wilairatana et al. 2002; Wu 2002). Since the discovery of artemisinin, a number of semi-synthetic derivatives have been developed for specific applications in malaria treatment.

Malaria remains a serious health problem which affects over 400 million people, especially in Africa and Southeast Asia, causing the deaths in excess of 2 million each year. Increasing resistance of the malaria parasite, Plasmodium falciparum, towards current antimalarial drugs is a cause for concern. The future value of antimalarial drugs based on the artemisinin structure is illustrated by the development by Bayer AG of Artemisone, an artemisinin derivative reported to be 10-30 fold more active than artesunate, for which clinical trials are currently under way.

Artemisinin is produced in relatively small amounts of 0.01 to 1.5% dry weight, making it and its derivatives relatively expensive (Gupta et al. 2002). Several studies describe the chemical synthesis of the sesquiterpene, but none are an economical alternative for isolation of artemisinin from the plant (Yadav, Babu, & Sabitha 2003). Therefore a higher concentration in the plant or production in an alternative host is desirable to make artemisinin available as economically as possible, especially for use in the Third World. Knowledge of the biosynthetic pathway and the genes involved should enable engineering of improved production of artemisinin. Alternatively, there is also the possibility of producing intermediates in the pathway to artemisinin which are of commercial value. For example, a compound 15 times more potent in vitro than artemisinin against Plasmodium falciparum has been synthesized from artemisinic alcohol (Jung, Lee, & Jung 2001).

There is evidence that artemisinin is localized to glandular trichomes on the surfaces of certain tissues of the plant (Duke et al. 1994; Duke & Paul 1993). The number and even existence of these trichomes and the amount of artemisinin varies widely among biotypes.

Typically, compounds discovered in plants and found to be useful are produced commercially by i) chemical synthesis, where possible and economical, ii) extraction of cultivated or wild plants, or iii) cell or tissue culture (this is rarely economical). In those cases in which chemical synthesis is not economical, it makes sense to learn as much as possible about the biosynthesis of a natural product, such that it can be produced most efficiently in plants or cell/tissue culture. In the case of artemisinin, chemical synthesis is not commercially feasible. Since the compound is produced in small quantities in Artemisia, the drugs derived from artemisinin are relatively expensive, particularly for the Third World countries in which they are used. While the antimalarial drugs, chloroquine and sulfadoxine-pyrimethamine, cost as little as 20 cents for an adult treatment, artemisinin-derived compounds, by contrast, can be 100 times as expensive. Chloroquine resistance is prevalent and sulfadoxine-pyrimethamine resistance is increasing. The World Health Organization recently added the artemisinin-derived drug, artemether to their Model List of Essential Medicines, which are recommended to be available at all times in adequate amounts and in the appropriate dosage forms, and at a price that individuals and the community can afford. Consequently, it would be useful to be able to supply artemisinin-derived drugs more economically.

There are numerous patents relating to artemisinin and artemisinin derived drugs. These cover drug synthesis and formulation, Artemisia cultivation (Kumar 2002) and tissue culture and artemisinin extraction (Elferaly 1990). In the past five years a reasonably clear picture of artemisinin biosynthesis has emerged as illustrated in FIG. 1 (Bertea et al. 2005). The identity of amorpha-4,11-diene as a biosynthetic intermediate was established, based on the presence of trace of amorpha-4,11-diene in Artemisia extracts and the cloning and expression of cDNAs representing amorpha-4,11-diene synthase, a terpene cyclase (Bouwmeester et al. 1999; Wallaart et al. 2001). A cytochrome P450 gene designated cyp71av1 was recently cloned and characterized (Teoh et al. 2006). The cyp71av1 gene encodes a hydroxylase that catalyzes the conversion of amorpha-4,11-diene to artemisinic alcohol. CYP71AV1 expressed in yeast is also capable of oxidizing artemisinic alcohol to artemisinic aldehyde and artemisinic aldehyde to artemisinic acid.

Commonly owned U.S. patent application 60/729,210 filed Oct. 24, 2005, the disclosure of which is herein incorporated by reference, and now filed as a PCT patent application published May 3, 2007 under publication number WO 2007/048235, discloses a gene encoding amorpha-4,11-diene hydroxylase, which catalyzes the first committed steps in artemisinin biosynthesis (FIG. 1).

Commonly owned international patent application PCT/CA2007/000614 filed Apr. 4, 2007 and published on Oct. 11, 2007 under publication number WO 2007/112596 discloses an artemisinic aldehyde double bond reductase and gene encoding the reductase. The artemisinic aldehyde double bond reductase disclosed in this publication reduces artemisinic aldehyde to dihydroartemisinic aldehyde, however, improvements to the stereospecificity of the reduction reaction would be desirable.

There is a need for an enzyme that provides improved stereospecific reduction of artemisinic aldehyde to dihydroartemisinic aldehyde.

### SUMMARY OF THE INVENTION

The invention described herein addresses the production of artemisinin and artemisinin-related compounds, including precursors, of pharmaceutical and commercial interest.

There is provided an isolated nucleic acid molecule encoding an artemisinic aldehyde double bond reductase, the isolated nucleic acid molecule comprising a nucleotide sequence having at least 70% sequence identity to the nucleotide sequence as set forth in nucleotides 63 to 1226 of SEQ ID No: 1.

There is provided a purified or partially purified artemisinic aldehyde double bond reductase comprising an amino acid sequence having at least 70% sequence identity to the amino acid sequence as set forth in SEQ ID No.: 2.

There is provided a purified or partially purified artemisinic aldehyde double bond reductase encoded by a nucleic acid molecule of the present invention.

There is provided a use of one or more purified or partially purified artemisinic aldehyde double bond reductases of the present invention in the production of dihydroartemisinic aldehyde, dihydroartemisinic acid and/or artemisinin acid.

There is provided a process for producing dihydroartemisinic aldehyde, dihydroartemisinic acid and/or artemisinin comprising expressing or overexpressing one or more isolated nucleic acid molecules of the present invention in a host cell.

There is provided a process for producing dihydroartemisinic aldehyde, dihydroartemisinic acid and/or artemisinin comprising producing or overproducing an artemisinic aldehyde double bond reductase of the present invention in a host cell.

There is provided a method of selecting or developing plants with altered dihydroartemisinic aldehyde, dihydroartemisinic acid, artemisinic acid and/or artemisinin levels in a population of plants that naturally produces dihydroartemisinic aldehyde, dihydroartemisinic acid, artemisinic acid and/or artemisinin comprising: detecting a target plant having altered levels of dihydroartemisinic aldehyde, dihydroartemisinic acid, artemisinic acid and/or artemisinin compared to a control plant provided under similar conditions; isolating at least a portion of an artemisinic aldehyde double bond reductase gene of the target plant and comparing the nucleotide sequence of said at least a portion to SEQ ID No.: 2 to detect a variation from SEQ ID No. 2; detecting the variation in other plants; selectively breeding the plants with the variation to produce a population of plants having altered levels of dihydroartemisinic aldehyde, dihydroartemisinic acid, artemisinic acid and/or artemisinin compared to a population of control plants produced under similar conditions.

There is provided a method of increasing dihydroartemisinic aldehyde, dihydroartemisinic acid, artemisinic acid and/or artemisinin levels in a population of plants that naturally produces dihydroartemisinic aldehyde, dihydroartemisinic acid, artemisinic acid and/or artemisinin comprising: providing a population of mutated plants; detecting a target mutated plant within the population of mutated plants, the target mutated plant having an altered expression of an artemisinic aldehyde double bond reductase gene or altered activity of an artemisinic aldehyde double bond reductase enzyme compared to a control plant provided under similar conditions, said detecting comprising using primers developed from a nucleic acid molecule of the present invention to PCR amplify regions of the artemisinic aldehyde double bond reductase gene from mutated plants in the population of mutated plants, identifying mismatches between the amplified regions and corresponding regions in wild-type gene that lead to the altered expression or altered activity, and identifying the mutated plant that contains the mismatches; and, selectively breeding the target mutated plant to produce a population of plants having altered expression of artemisinic aldehyde double bond reductase gene or altered activity of artemisinic aldehyde double bond reductase enzyme compared to a population of control plants produced under similar conditions.

The artemisinic aldehyde double bond reductase of the present invention provides improved stereospecific reduction of artemisinic aldehyde to biologically active dihydroartemisinic aldehyde than artemisinic aldehyde double bond reductases of the prior art.

The nucleotide sequence may have at least 80%, at least 90%, at least 95% or at least 99% sequence identity to the nucleotide sequence as set forth in nucleotides 63 to 1226 of SEQ ID No: 1. The amino acid sequence may have at least 80%, at least 90%, at least 95% or at least 99% sequence identity to the amino acid sequence as set forth in SEQ ID No.: 2.

The gene (nucleic acid molecule) of the present invention may be derived, for example cloned, from Artemisia annua. Obtaining other nucleic acid molecules of the present invention may be accomplished by well-known techniques in the art. Such techniques are disclosed in Sambrook et al. 2001 and Ausubel et al. eds. 2001. Generally, other nucleic acid molecules of the present invention may be obtained by: a) identifying the existence of a homologous gene by techniques such as, for example, hybridization of a target gene to the complement of SEQ ID No: 1, genome or transcriptome (cDNA) sequencing, or database searching in nucleic acid sequence databases such as Genbank; b) cloning the homologous gene and creating a plasmid for E. coli or yeast expression as described in Sambrook et al. 2001 or Ausubel et al. eds. 2001; and, c) then testing the gene product for artemisinic aldehyde reduction as described herein below. Database searching may employ commonly used computer programs such as BLASTX, BLASTP, TBBLASTN and others.

Other nucleic acid molecules and proteins of the present invention may also be obtained by creating mutations of nucleic acid molecules and proteins already at hand. Such mutations may be accomplished by commonly known methods in the art as described in Sambrook et al. 2001 and Ausubel et al. eds. 2001.

Overexpression of one or more of the nucleic acid molecules or overproduction of the artemisinic aldehyde double bond reductase may be done in A. annua. Expression of one or more of the nucleic acid molecules or expression of the artemisinic aldehyde double bond reductase may be done in other hosts, for example plants, yeasts or bacteria. Overexpression or expression of one or more of the isolated nucleic acid molecules of the present invention may be done in combination with overexpression or expression of one or more other nucleic acid molecules involved in the biosynthesis of artemisinin, for example those encoding farnesyl diphosphate synthase, amorpha-4,11-diene synthase, amorpha-4,11-diene hydroxylase, alcohol dehydrogenase, aldehyde dehydrogenase.

Part of the solution to the problem of producing artemisinin in an economical and timely fashion is the isolation and exploitation of genes involved in artemisinin biosynthesis. As in other examples of metabolic engineering, such genes can be used to enhance production by overexpression in the native plant (A. annua), a different plant, or in micro-organisms such as bacteria or yeast. An example of this is the expression of the amorphadiene synthase gene in E. coli to produce the artemisinin precursor amorphadiene (Martin et al. 2003) and the production of artemisinic acid in yeast (Ro et al. 2006). One important step in the pathway to artemisinin per se, is thought to be the reduction of artemisinic aldehyde to (11R)-dihydroartemisinic aldehyde. Consequently, the genes involved in this step may be used to produce dihydroartemisinic aldehyde and/or (11R)-dihydroartemisinic acid in a host, alone or in combination with one or more of farnesyl diphosphate synthase, amorpha-4,11-diene synthase, amorpha-4,11-diene hydroxylase, alcohol dehydrogenase and aldehyde dehydrogenase (for example, the artemisinic/dihydroartemisinic aldehyde dehydrogenase gene disclosed in WO 2007/112596 published Oct. 11, 2007).

The resulting (11R)-dihydroartemisinic acid could then be chemically converted to artemisinin or related compounds of commercial value. Dihydroartemisinic acid is a presumed late precursor of artemisinin, and its transformation to artemisinin has been shown to occur spontaneously through photo-oxidation, requiring no enzyme intervention (Sy & Brown 2002; Wallaart et al. 1999). Consequently, using (11R)-dihydroartemisinic acid instead of artemisinic acid as the starting material for semi-synthesis of artemisinin reduces the number of steps required for artemisinin production thus, simplifying the production process. This may lead to shorter artemisinin production time and lower production cost. The eventual outcome will be cheaper artemisinin and artemisinin-related drugs.

Nucleic acid molecules of the present invention may also be used in the development of DNA markers and in targeted mutagenesis techniques (e.g. TILLING (Targeting Induced Local Lesions IN Genomes)).

A genetic marker (DNA marker) is a segment of DNA with an identifiable physical location on a chromosome and associated with a particular gene or trait and whose inheritance can be followed. A marker can be a gene, or it can be some section of DNA with no known function. Because DNA segments that lie near each other on a chromosome tend to be inherited together, markers are often used as indirect ways of tracking the inheritance pattern of a gene that has not yet been identified, but whose approximate location in the genome is known. Thus, markers can assist breeders in developing populations of organism having a particular trait of interest. Gene-specific markers can be used to detect genetic variation among individuals which is more likely to affect phenotypes relating to the function of a specific gene. For example, variation in a gene-specific marker based on AaDBR2, rather than variation in an anonymous DNA marker, would be more likely linked to variation in content of artemisinin or related compounds, by virtue of its association with the relevant biosynthetic pathway. In one embodiment, a DNA marker for AaDBR2 could be developed by sequencing the polymerase chain reaction amplified AaDBR2 gene from a number of individual plants of Artemisia annua. Such sequencing would provide information about sequence polymorphisms within the gene. A range of methods available to those skilled in the art could be used to detect such polymorphisms, including cleaved amplified polymorphic sequences (CAPs) (Konieczny & Ausubel 1993).

The presence of such gene-specific polymorphisms could be correlated with levels of artemisinin or related compounds and used in a breeding program to select and/or develop lines of Artemisia annua with enhanced levels of artemisinin or related compounds. That is, the variation in genetic structure may be detected in other plants, and the plants with the variation selectively bred to produce a population of plants having increased levels of dihydroartemisinic aldehyde, dihydroartemisinic acid artemisinic acid and/or artemisinin compared to a population of control plants produced under similar conditions. Genetic markers are discussed in more detail in Bagge et al. 2007, Pfaff et al. 2003, Sandal et al. 2002 and Stone et al. 2002.

TILLING (Bagge, Xia, & Lubberstedt 2007; Comai & Henikoff 2006; Henikoff, Till, & Comai 2004; Slade & Knauf 2005) involves treating seeds or individual cells with a mutagen to cause point mutations that are then discovered in genes of interest using a sensitive method for single-nucleotide mutation detection. Detection of desired mutations (e.g. mutations resulting in a change in expression of the gene product of interest) may be accomplished, for example, by PCR methods. For example, oligonucleotide primers derived from the gene (nucleic acid molecule) of interest, such as the nucleic acid molecules of the present invention, may be prepared and PCR may be used to amplify regions of the gene of interest from plants in the mutagenized population. Amplified mutant genes may be annealed to wild-type genes to find mismatches between the mutant genes and wild-type genes. Detected differences may be traced back to the plants which had the mutant gene thereby revealing which mutagenized plants will have the desired expression. These plants may then be selectively bred to produce a population having the desired expression.

Further features of the invention will be described or will become apparent in the course of the following detailed description.

### BRIEF DESCRIPTION OF THE DRAWINGS

In order that the invention may be more clearly understood, embodiments thereof will now be described in detail by way of example, with reference to the accompanying drawings, in which:

FIG. 1 depicts the proposed biosynthetic pathway for artemisinin biosynthesis.

FIG. 2 depicts the nucleotide sequence (SEQ ID No.: 1) of the AaDBR2 cDNA encoding artemisinic aldehyde double bond reductase.

FIG. 3 depicts the predicted amino acid sequence (SEQ ID No.: 2) of the protein encoded by AaDBR2.

FIG. 4 depicts the nucleotide sequence (SEQ ID No.: 3) of the open reading frame of the AaDBR2DNA insert in pDEST17-AaDBR2.

FIG. 5 depicts the predicted amino acid sequence (SEQ ID No.: 4) of the product of the AaDBR2 insert in pDEST17 in frame with an N-terminal His tag sequence.

FIG. 6 depicts a GC/MS analysis of double bond reductase assays using artemisinic aldehyde as the substrate. Artemisia annua flower bud protein extracts were assayed without NADPH (a) and with NADPH (b). AaDBR2 was purified from E. coli and assayed without NADPH (c) with NADPH (d). The retention times and mass spectra of the peaks at 13.88 min are equivalent to the standard (11R)-dihydroartemisinic aldehyde (Mᵣ⁺ 219).

FIG. 7 depicts a GC/MS analysis of the extracts from yeast strains containing all three empty vectors pESC-HIS, pESC-LEU, and pYESDEST52 (a), the plasmids pESC-HIS-FPS-ADS, pESC-LEU-CYP-CPR, and pYESDEST52 (b), and the plasmids pESC-HIS-FPS-ADS, pESC-LEU-CYP-CPR, and pYESDEST52-AaDBR2. Artemisinic acid (AA) was formed in the yeast strain expressing CYP71AV1 (b and c). (11R)-Dihydroartemisinic acid (DHAA) was formed in the yeast strain which additionally expressed AaDBR2 and was detected as methyl (11R)-dihydroartemisinate (c). The retention time and mass spectrum of the peak at 14.77 min are equivalent to the standard methyl (11R)-dihydroartemisinate (Mᵣ 250). Chromatograms correspond to equivalent volumes of yeast culture.

FIG. 8 depicts chromatograms of products of AaDBR2 catalyzed reactions. GC/MS analysis is shown of AaDBR2 products of artemisinic aldehyde, 2E-nonenal (+)-carvone and 2-cyclohexen-1-one in the absence (A, C, E and G, respectively) and presence (B, D, F and H, respectively) of NADPH. AAA=artemisinic aldehyde. DHAAA=(11R)-dihydroartemisinic aldehyde.

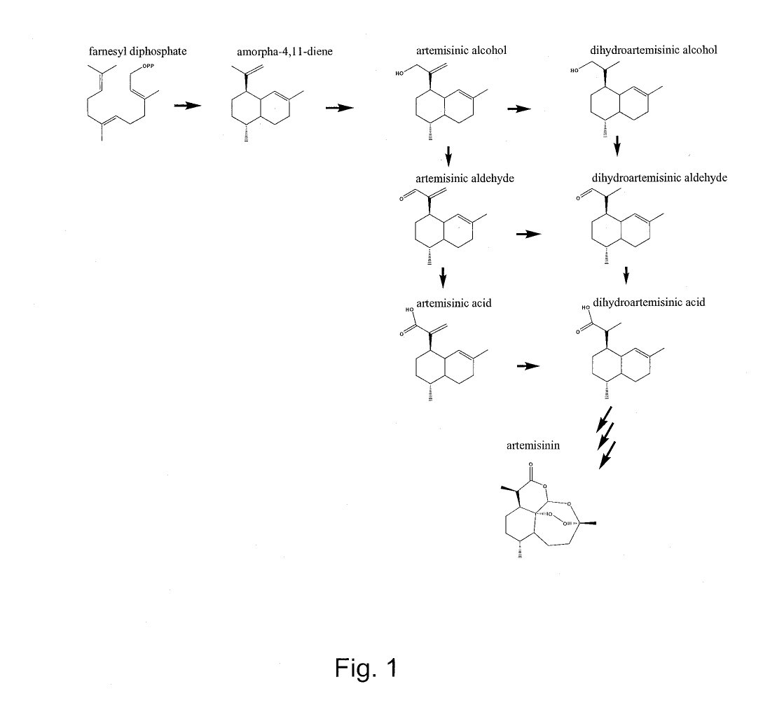

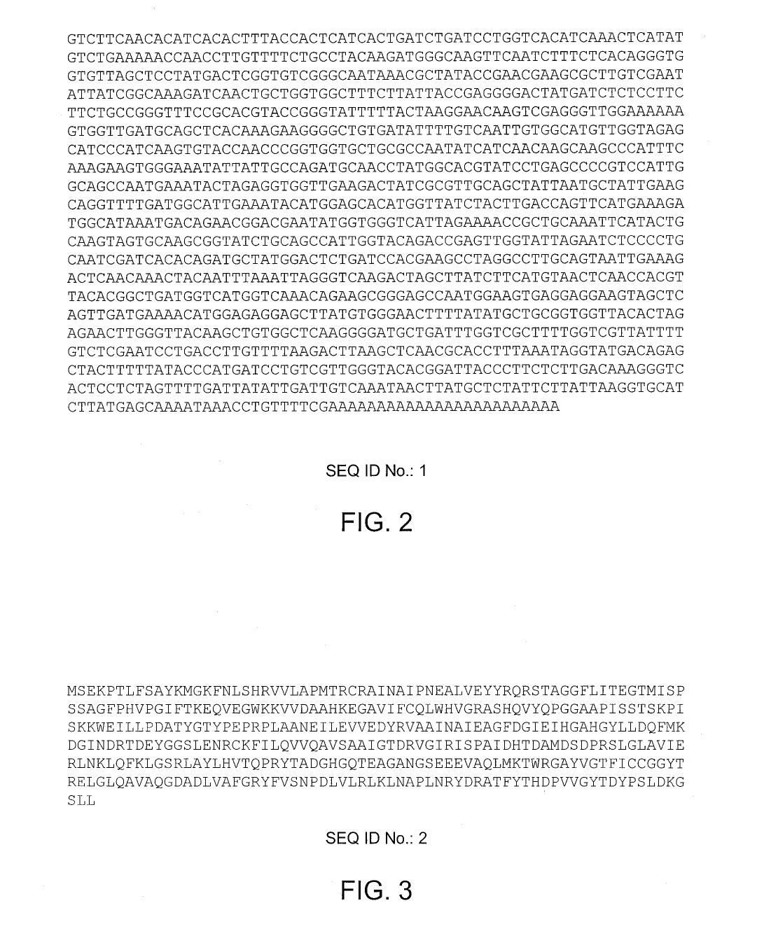

### DESCRIPTION OF PREFERRED EMBODIMENTS

### Materials and Methods

### Artemisinic Aldehyde

Artemisinic acid was isolated from dichloromethane extracts of A. annua flower buds and leaves (Teoh, Polichuk, Reed, Nowak, & Covello 2006) and was used to synthesize artemisinic aldehyde according to the method described by Chang et al. 2000, the disclosure of which is incorporated herein by reference.

### Dihydroartemisinic Acid

Dihydroartemisinic acid was isolated and purified from A. annua leaf material obtained from a “line 2/39” containing relatively high levels of the dihydroartemisinic acid using the method described for artemisinic acid in Teoh et al. 2006, the disclosure of which is incorporated herein by reference.

### Dihydroartemisinic Aldehyde

Dihydroartemisinic aldehyde was synthesized from the isolated dihydroartemisinic acid (see above). The acid was converted to methyl dihydroartemisinate with excess diazomethane in diethyl ether at 0° C. for 5 minutes. The ether and diazomethane were removed under a stream of nitrogen and the methyl ester was reduced to (11R)-dihydroartemisinic alcohol with excess 1.5 M diisobutyl aluminum hydride in toluene at room temperature for 10 min under nitrogen. With subsequent extraction, oxidation to the aldehyde with pyridinium chlorochromate (Corey & Suggs 1975) and purification by HPLC the (11R)-dihydroartemisinic aldehyde was produced at an overall yield of 48% with >90% purity according to GC analysis.

### Other Chemicals

Artemisinin, pyridinium chlorochromate, coniferyl aldehyde, 2-cyclohexen-1-one, 2E-hexenal, hexanal, 2E-nonenal, nonanal, (+)-α-pinene, and (+)-pulegone were obtained from Aldrich. (+)-Carvone, cyclohexanone, dihydrocarvone were obtained from Sigma. Arteannuin B and artemisitene were kindly provided by Dieter Deforce (University of Ghent). Artemisinic alcohol preparation was described previously (Teoh 2006). Sabinone was synthesized from sabinyl acetate obtained from the Plant Biotechnology Institute terpene collection (von Rudloff 1963) by saponification of the sabinyl acetate to d-sabinol followed by oxidation of the alcohol using pyridinium chlorochromate (Corey 1975).

### Plant Materials

Artemisia annua L. seeds were obtained from Elixir Farm Botanicals, Brixey, Mo., USA and from Pedro Melillo de Magalhães, State University of Campinas, Brazil (line 2/39). Seeds were germinated and grown in soil in a controlled environment chamber with 16 hour/25° C. days and 8 hour/20° C. nights. Plants that had reached the height of approximately 1.2 m (about 3 months) were transferred to flowering chamber with 12 hour/25° C. days and 12 hour/20° C. nights for the Elixir line and 8 hour/25° C. days and 16 hour/20° C. nights for line 2/39. Flower buds that developed after 19-21 days in the flowering chamber were harvested for total RNA isolation.

cDNA Library Construction and Expressed Sequence Tag (EST) Analysis

Total RNA was extracted and isolated from glandular trichomes and flower buds using a modified method described by Logeman, et al. 1987. cDNA synthesis from 1.5 micrograms of total RNA and construction of the trichome and flower bud cDNA library were carried out with Creator™ SMART™ cDNA Library Construction Kit (Clontech). A total of 6,239 clones and 2,208 clones for trichome and flower bud libraries, respectively were randomly picked and their DNA sequences determined. Sequencing was performed on an AB13700 DNA sequencer using BigDye Terminator Cycle Sequencing Kit (Applied Biosystems) and the M13 reverse primer. DNA sequence traces were interpreted and vector and low quality sequences were eliminated using PHRED (Ewing et al. 1998) and LUCY (Chou & Holmes 2001). Clustering of the resulting EST dataset was done using STACKPACK (Miller et al. 1999) and sequence similarity was identified by BLAST (Altschul et al. 1990).

Preparation of the Clarified Extracts Containing Artemisinic Aldehyde Double Bond Reductase from Artemisia Annua

All of the operations were carried out at 4° C. Twenty four grams of flower buds from line 2/39 were ground in a Waring blender with 100 mL of 0.1 M potassium phosphate (pH 7.0) containing 5 mM dithiothreitol. The resulting slurry was filtered through three layers of cheesecloth. The filtrate was centrifuged at 10,000×g for 15 min. The supernatant was precipitated in a step-wise fashion by the addition of (NH₄)₂SO₄. After removal of the precipitate at 30% (NH₄)₂SO₄ saturation, the precipitate of the supernatant at 80% (NH₄)₂SO₄ saturation was collected by centrifugation. The pellet was dissolved in 3 mL of 20 mM Tris-HCl buffer (pH 7.3) and centrifuged for 10 min at 10,000×g. The clear supernatant was dialyzed against a Tris-HCl buffer (pH 7.3).

At various stages of purification double bond reductase assays were performed on plant extracts with artemisinic aldehyde, followed by gas chromatography/mass spectrometry analysis. Enzyme reactions were initiated by adding the 0.4 mM artemisinic aldehyde to 300 μL reaction mixture containing plant extract (10-300 μg protein), 50 mM Tris-HCl (pH 8.0), 1 mM NADPH and 2 mM DTT. Negative controls were carried out with boiled proteins and without NADPH. Reactions were allowed to proceed for 30 minutes at 30° C. with shaking, stopped by the addition of 15 μl of acetic acid and extracted with 100 μL ethyl acetate. The ethyl acetate extracts were then subjected to GC/MS analysis. The reaction products were confirmed by comparing GC retention time and MS data with those of synthetic (11R)-dihydroartemisinic aldehyde. For quantification, octadecane was used as an internal standard.

Partial Purification of the Artemisinic Aldehyde Double Bond Reductase from Artemisia Annua

The aforementioned dialyzed extract was applied to a Mono-Q HR strong anion ion exchange column (5×50 mm; GE Healthcare Life Sciences) pre-equilibrated with 10 mM potassium phosphate buffer (pH 7.8) containing 1 mM DTT and eluted with 30 mL of a linear KCl gradient (0-0.5 M) in 10 mM potassium phosphate buffer (pH 7.8) at a flow rate of 1.0 mL/min. One mL fractions were collected and tested for the artemisinic aldehyde double bond reductase activity as follows. Each fraction was desalted by spin dialysis (Amicon Ultra-15 devices; Millipore, Mass.) into assay buffer (50 mM potassium phosphate buffer, pH 7.5) containing 1 mM DTT. Reactions were initiated with the addition of 1 mM NADPH and 0.4 mM artemisinic aldehyde in a total volume of 100 μL, and allowed for proceed at 30° C. for 30 min prior to the addition of 50 μL ethyl acetate. Thirty μL of ethyl acetate extracts from the reactions were used for GC-MS analysis. The reaction products were confirmed by comparing GC retention time and MS data those of synthetic (11R)-dihydroartemisinic aldehyde. For quantification octadecane was used as an internal standard. The active fractions were combined, desalted and concentrated by spin dialysis (Amicon Ultra-15 devices; Millipore, Mass.). The combined sample was loaded onto a Superose™ 6 (10×300 mm; GE Healthcare Life Sciences) equilibrated with 10 mM potassium phosphate buffer (pH 7.8) containing 100 mM KCl. Protein was eluted at a flow rate of 1 mL/min. Fractions of 1 mL were collected and tested for artemisinic aldehyde double bond reductase activity (see above). Retention times were compared with those of the following gel filtration markers (GE Healthcare Life Sciences): thyroglobulin (669 kDa), ferritin (440 kDa), catalase (232 kDa), aldolase (158 kDa), BSA (67 kDa), ovalbumin (43 kDa), chymotrypsinogen (25 kDa), and ribonuclease A (14 kDa). Elution was monitored at 280 nm. The molecular mass in the native state of the artemisinic alcohol double bond reductase was estimated to be 44 kDa. The active fractions were combined, desalted by dialysis with 10 mM potassium phosphate containing 1 mM DTT and concentrated by ultrafiltration for subsequent purification using a batch affinity purification step as follows. Reactive Red 120 Agarose (Type 3000-CL, Sigma; 100 μl) was pre-equilibrated with an equal volume of 10 mM potassium phosphate buffer (pH 7.8) containing 2 mM ZnCl₂. The buffer was removed and 500 μl of protein solution was added and gently shaken on ice for 10 min. The buffer was removed and the agarose was washed with 3×1 ml 10 mM potassium phosphate containing 1 mM DTT.

The active protein was eluted by incubating on ice with 3×1 ml 10 mM potassium phosphate containing 1 mM DTT, 1 mM EDTA and 1 mM NADPH. The eluted protein was desalted and concentrated by spin dialysis (Amicon Ultra-15 devices; Millipore, Mass.).

### In-Gel Trypsin Digestion

The double bond reductase preparation was subjected to SDS-PAGE (10-20% acrylamide) under reducing conditions followed by silver staining. The bands of interest were excised from the gel. In-gel trypsin digestion was performed according to the method of MassPREP Digestion 5.0 (Waters MassPREP™ Station) as follows. The gel pieces were de-stained twice with a solution containing 30 mM potassium ferricyanide, 100 mM sodium thiosulphate, 100 mM ammonium bicarbonate and 50% (v/v) acetonitrile, then reduced with 10 mM DTT, followed by alkylation with 55 mM iodoacetamide. The gel pieces were destained two more times and rehydrated in digestion buffer containing 100 mM ammonium bicarbonate and 6 ng/μl trypsin (sequencing grade, Promega). After 5 h incubation at 37° C., the gel slices were extracted three times with 1% (v/v) formic acid and 2% (v/v) acetonitrile. The gel extracts were transferred to a 96 well PCR plate, using a MassPREP protein digest station (Wates/Micromass, Manchester, UK) prior to LC-MS analysis.

### MALDI-TOF Mass Spectrometry Analysis of the Digested Peptides

The aforementioned gel extracts containing trypsin-digested proteins were evaporated to dryness, then dissolved in 6 μl of 1% (w/v) aqueous trifluoroacetic acid (TFA). The resulting solutions were subjected to LC-MS by injection into a NanoAcquity HPLC (Waters, Milford, Mass., USA) interfaced to a Q-ToF Ultima Global hybrid tandem mass spectrometer fitted with a Z-spray nanoelectrospray ion source (Waters/Micromass, Manchester, UK). Solvent A consisted of 0.1% (v/v) formic acid in water, while solvent B consisted of 0.1% (v/v) formic acid in acetonitrile. The samples were first adsorbed on a C18 trapping column (Symmetry 180 μm×20 mm; Waters) and washed for 3 min using solvent A at a flow rate of 15 μL/min. The trapped peptides were eluted onto a C18 analytical column (1.7 μm BEH130 C18 100 μm×100 mm; Waters). Separations were performed using a linear gradient of 10:90% to 45:55% A:B over 45 min. The composition was then changed to 20:80% A:B and held for 10 min to flush the column before re-equilibrating for 7 min at 100% Solvent A. Mass calibration of the Q-ToF instrument was performed using a product ion spectrum of Glu-fibrinopeptide B acquired over the m/z range 50 to 1900. LC-MS/MS analysis was carried out using data dependent acquisition, during which peptide precursor ions were detected by scanning from m/z 400 to 1900 in TOF MS mode. Multiply charged (2+, 3+, or 4+) ions rising above predetermined threshold intensity were automatically selected for TOF MS/MS analysis, by directing these ions into the collision cell where they fragment using low energy collision-induced dissociation (CID) by collisions with argon and varying the collision energy by charge state recognition, product ion spectra were acquired over the m/z range 50 to 1900. LC-MS/MS data was processed using Mascot Distiller ver. 2.1.1.0; Matrixscience) used to searched a local Artemisia annua expressed sequence tag database (Teoh, Polichuk, Reed, Nowak, & Covello 2006) using MASCOT (Matrix Science Inc., Boston, Mass.). Searches were performed using carbamidomethylation of cysteine as a fixed modification and oxidation of methionine as a variable modification, allowing for one missed cleavage during trypsin digestion.

Isolation of Full-Length AaDBR2 cDNA

A cDNA encoding the Artemisia annua artemisinic aldehyde double bond reductase designated AaDBR2, and corresponding to the EST clone GSTSUB 026_G01, was obtained through SMART-RACE-PCR using gene-specific primers 5′-GCTCATAAGATGCACCTTAATAAG-3′ (SEQ ID No.: 5) and adaptor PCR primer 5′-AAGCAGTGGTATCAACGCAGAGT-3′ (SEQ ID No.: 6) (Clontech Laboratories, Inc.) and Taq DNA polymerase (Invitrogen Canada Inc.). The resulting PCR product was cloned into PCR 2.1-TOPO vector resulting in the plasmid the pPCR2.1-AaDBR2. The DNA sequence of the insert of PCR2.1-AaDBR2 was determined with an AB13700 DNA sequencer using a BigDye Terminator Cycle Sequencing Kit (Applied Biosystems).

### Expression in E. Coli

For E. coli expression, the open reading frame (ORF) of the AaDBR2 gene was PCR-amplified using gene-specific primers 5′-CACCATGTCTGAAAAACCAACCTTG-3′ (SEQ ID No.: 7) and 5′-GCTCATAAGATGCACCTTAATAAG-3′ (SEQ ID No.: 8), Vent DNA polymerase (New England BioLabs, Cambridge, Mass., USA) and the plasmid of pPCR2.1-AaDBR2 as the template. The resulting PCR product was cloned via the Gateway entry vector pENTR/D/TOPO (Invitrogen) into the Gateway destination vector pDEST17 (Invitrogen) to generate a bacterial expression clone pDEST17-AaDBR2. The ORF of AaDBR2 was in frame with vector sequence encoding an N-terminal His₆ tag. The plasmid pDEST17-AaDBR2 was introduced into E. coli strain Rosetta™ 2(DE3) (Novagen) using heat shock at 42 C. Transformants were grown on Luria Broth (LB) and selected on ampicillin (100 μg/mL) at 37° C. for 24 hours. A single colony containing pDEST17-AaDBR2 was used to inoculate 5 mL of LB liquid medium with ampicillin (LBA) and grown at 37° C. overnight with shaking. The overnight culture was used to inoculate 250 mL of LBA liquid medium and grown at 37° C. with shaking to an OD₆₀₀ of 0.6 per mL followed by induction with 1 mM IPTG and grown at 30° C. for 4 hours with shaking. Cells were centrifuged at 2,000×g at 4° C. for 10 minutes. The resulting cell pellets were resuspended in 6 mL of lysis buffer consisting of 50 mM sodium phosphate (pH 8.0), 0.1 M NaCl, 20 mM imidazole and 1 mM phenylmethylsulfonyl fluoride (PMSF). Cells were lysed with lysozyme (0.2 mg/mL of cell suspension) on ice for 30 minutes followed by sonication on ice with five 30 s pulses. The sonicated E. coli extract was then centrifuged at 20,000×g for 15 min at 4° C. to obtain the supernatant as a cell-free extract containing AaDBR2.

### Purification of Recombinant AaDBR2

The aforementioned cell-free extract was loaded onto a His-Trap FF column (Amersham Bioscience, NJ) equilibrated with binding buffer (20 mM sodium phosphate buffer containing 500 mM NaCl and 20 mM imidazole at pH 7.5). The column was washed with 5 column volumes of binding buffer and the recombinant AaDBR2 was eluted with elution buffer (20 mM sodium phosphate, 500 mM NaCl, pH 7.5) containing increasing concentrations of imidazole in a step-wise fashion (50 mM, 100 mM, 200 mM, 250 mM, and 300 mM imidazole). Most of AaDBR2 was eluted in the elution buffer containing 200 mM imidazole. The eluted fractions were concentrated and desalted by spin dialysis (Amicon Ultra-15 devices; Millipore, Mass.) following manufacturer's protocol. The purity of the recombinant AaDBR2 was checked by SDS-PAGE using Rapid Stain (Biosciences, St. Louis, Mo.) for visualization.

In Vitro Enzyme Assays with Artemisinic Aldehyde as Substrate

The purified recombinant His-tagged AaDBR2 protein was assayed with artemisinic aldehyde, followed by gas chromatography/mass spectrometry analysis. Enzyme reactions were initiated by adding the 0.4 mM artemisinic aldehyde to 300 μL reaction mixture containing 50 mM Tris-HCl (pH 8.0), 1 mM NADPH, 2 mM DTT and 2.0 μg of AaDBR2. Negative controls were carried out with boiled proteins, without NADPH. Reactions were allowed to proceed for 30 minutes at 30° C. with shaking, stopped by adding 15 μL acetic acid and extracted with 100 μL ethyl acetate. The ethyl acetate extracts were pooled and partially evaporated prior to GC-MS analysis. The reaction products were confirmed by comparing GC retention time and MS data with those of synthetic (11R)-dihydroartemisinic aldehyde. For quantification, octadecane was used as an internal standard.

In Vitro Enzyme Assays with Other Substrates

For comparison of the catalytic properties of AaDBR2 against other substrates, reaction mixtures were pre-warmed to 30° C., and reactions were initiated by addition of AaDBR2. The pH optimum of the purified AaDBR2 was determined to be 7.5 in assay that included one of three 50 mM buffers (MES, HEPES, and Tris-HCl) adjusted to between pH 5.5 and 9.0 in 0.5-unit intervals and 0.5 mM artemisinic aldehyde and 0.48 μg of purified recombinant AaDBR2. Apparent kinetic parameters were determined under conditions that limited conversion to less than 10% as follows. Concentrations of artemisinic aldehyde (6-250 μM, 0.24 μg of AaDBR2, 2 min), 2-cyclohexen-1-one (80-10,000 μM, 0.48 μg of AaDBR2, 10 min), and (+)-carvone (20-5000 μM, 0.48 μg AaDBR2, 10 min) were varied in the presence of 1 mM NADPH. To assess cofactor specificity, concentrations of NADPH (10-640 μM, 0.48 μg of AaDBR2, 2 min) and NADH (0.2-13 mM, 1.9 μg of AaDBR2, 2 min) were varied in the presence of 0.5 mM artemisinic aldehyde. The ethyl acetate extracts from triplicate reaction mixtures were directly analyzed by GC/MS. Octadecane was used as an internal standard to quantify the products formed from the reactions using response factors determined using standards for each enzyme product. Kₘ and k_{cat} values were determined by nonlinear regression analysis using GraphPad™ software (GraphPad™ Software Inc., San Diego, Calif.).

### Yeast Expression Plasmids

As a means of testing the effect of expression of multiple genes in the artemisinin pathway in yeast, the plasmids pESC-HIS-FPS-ADS, pESC-LEU-CYP-CPR and pYES-DEST52-AaDBR2 were constructed as follows. All plasmids were confirmed by DNA sequencing.

The open reading frame of farnesyl pyrophosphate synthase (FPS; GenBank accession No. AF136602) was isolated from A. annua plants by RT-PCR using the oligonucleotide primers 5′-TAAGCGGCCGCATGAGTAGCATCGATCTGAAATCC-3′ (SEQ ID No.: 9), and 5′-TAAACTAGTCTACTTTTGCCTCTTGTAGATTT-3′ (SEQ ID No.: 10). The underlined sequences denote NotI and SpeI restriction sites for sub-cloning, and the start and stop codons of the ORF are indicated bold. The resulting PCR product was digested with NotI and SpeI and ligated into the yeast expression vector pESC-HIS (Stratagene, La Jolla, Calif.) to give the plasmid pESC-HIS-FPS.

Similarly, the open reading frame of amorpha-4,11-diene synthase (ADS; GenBank accession No. AF138959), was amplified by RT-PCR using the oligonucleotide primers 5′-TAAGGATCCATGGAACAGCAACAAGAAGTGATC-3′ (SEQ ID No.: 11) and 5′-TAAGGGCCCTCTATACTCATAGGATAAACGAG-3′ (SEQ ID No.: 12). The BamHI- and ApaI-digested PCR product was ligated into the BamHI- and an Apa1-digested plasmid pESC-HIS-FPS to give the plasmid pESC-HIS-FPS-ADS. In this construct, the ADS gene was fused with a myc tag.

The open reading frames of CYP71AV1 (GenBank accession No. DQ315671) and A. annua cytochrome P450 reductase (CPR; GenBank accession No. EF104642) were amplified by RT-PCR. For PCR-amplification of CYP71AV1, oligonucleotide primers 5′-ATTGGATCCATGAAGAGTATACTAAAAG-CAATG-3′ (SEQ ID No.: 13) and 5′-TAAGTCGACCTAGAAACTTGGAACGAGTAACAAC-3′ (SEQ ID No.: 14) were used; for PCR-amplification of CPR, oligonucleotide primers 5′-ATTGCGGCCGCATGCAATCAACAACTTCCGTTAAG-3′ (SEQ ID No.: 15) and 5′-TGATTAATTAATTACCATACATCACGGAGATATC-3′ (SEQ ID No.: 16) were used. The BamHI- and SalI-digested CYP71AV1 PCR product was ligated into the BamHI- and an SalI-digested plasmid pESC-LEU (Stratagene) to give the plasmid pESC-LEU-CYP. The NotI- and PacI-digested CPR PCR product was ligated into the NotI- and PacI-digested plasmid pESC-LEU-CYP to give the plasmid pESC-LEU-CYP-CPR.

The AaDBR2 ORF in pENTR/D-AaDBR2 was subcloned into the Gateway yeast expression vector pYES-DEST52 (Invitrogen) to generate a yeast expression construct pYES-DEST52-AaDBR2 through the recombination between the aforementioned pENTR/D-AaDBR2 and pYES-DEST52 by LR reaction (Invitrogen).

### Genetic Engineering of (11-R)-Dihydroartemisinic Acid Production in Yeast

The Saccharomyces cerevisiae strains (oye2 and oye3 deletion strains derived from the strain CY4) used in this study were provided by Dr. Chris M. Grant (Trotter et al. 2006). Competent cells of the oye2 and oye3 deletion strains were prepared with the S.c. EasyComp™ Transformation kit (Invitrogen) and co-transformed with pESC-HIS-FPS-ADS, pESC-LEU-CYP-CPR and either pYES-DEST52 (for vector control) or pYES-DEST52-AaDBR2 (for AaDBR2 co-expression).

For additional control experiments the empty yeast expression vectors pESC-HIS, pESC-LEU and pYES-DEST52 were co-transformed into the yeast cells. Yeast cultures (10 mL) were grown overnight at 30° C. in his, leu, ura liquid dropout medium (Clontech, Mountain View, Calif.) containing 2% (w/v) glucose. After 24 h in an orbital shaker maintained at 250 rpm, cells were collected by centrifugation and washed three times with sterile water. The cells were then resuspended to an OD₆₀₀ of 0.8 in his, leu, ura liquid dropout medium containing 2% (w/v) galactose, and grown for another 36 h.

Yeast cultures were then centrifuged and the medium was removed and extracted with 1 ml ethyl acetate. The yeast pellet was suspended in potassium phosphate buffer (pH 9.0) and sonicated in a sonicating water bath for 2 min. The suspension was centrifuged. The supernatant was removed, acidified with 1 ml of 2N HCl and extracted with 1 ml ethyl acetate. The ethyl acetate fractions from the medium and yeast were pooled and 20 μg of octadecane was added as an internal standard. The ethyl acetate extracts were concentrated to dryness under a nitrogen stream and methylated by treatment with diazomethane prior to GC-MS analysis.

### Results:

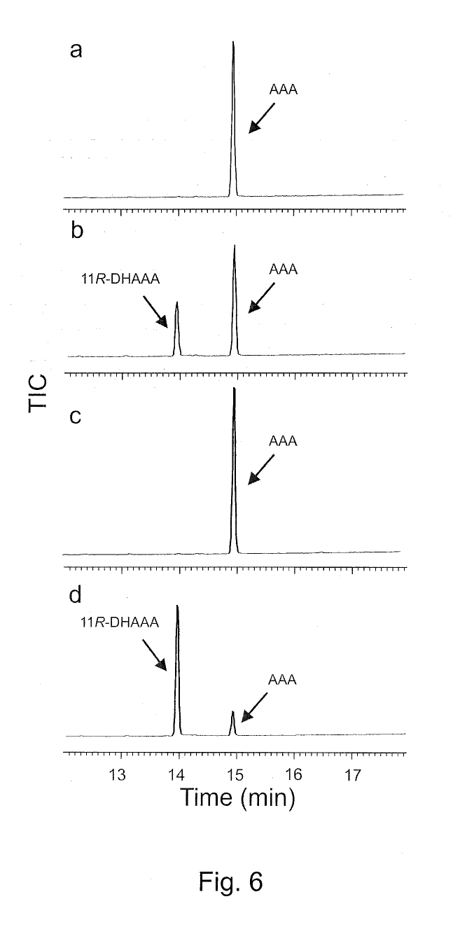

Crude enzyme extracts were initially prepared from the flower buds of A. annua line 2/39 and assayed for the conversion of artemisinic aldehyde to (11R)-dihydroartemisinic aldehyde. In this study, it was established that artemisinic aldehyde underwent reduction to afford (11R)-dihydroartemisinic aldehyde in the presence of NADPH (FIGS. 6, a and b). The enzyme responsible for the reaction, artemisinic aldehyde double bond reductase, was partially purified from the flower buds of A. annua line 2/39 using four steps that included fractional ammonium sulphate precipitation and ion-exchange, size exclusion and affinity chromatography. This resulted in a 1200-fold purification on a protein basis relative to the original plant extract. The partially purified enzyme was subjected to SDS-PAGE followed by silver staining. The most densely staining bands were excised from the polyacrylamide gel, digested with trypsin, and analyzed by LC/MS. When the resulting data from a band corresponding to 43 kDa was used to search a database of Artemisia annua expressed sequence tags (EST) a four peptide match was found to the EST called GSTSUB_{—}50_F07 as indicated in Table 1. Table 1 depicts the results of a MASCOT proteomics search showing five peptide signals from LC/MS/MS which match four peptide sequences predicted by the Artemisia annua expressed sequence tag named GSTSUB_{—}50_F07.

TABLE 1

| Observed | | | | | | |
|---|---|---|---|---|---|---|
| m/z | Mr(expt) | Mr(calc) | Delta | Score | Expect | Peptide |
| 479.3532 | 956.6918 | 956.5655 | 0.1263 | 48 | 0.00044 | R.SLGLAVIER.L |
| | | | | | | (SEQ ID No.: 17) |
| 599.4206 | 1196.8267 | 1196.6666 | 0.1601 | 49 | 0.00042 | K.LAYLHVTQPR.Y |
| | | | | | | (SEQ ID No.: 18) |
| 399.9546 | 1196.8421 | 1196.6666 | 0.1755 | (32) | 0.021 | K.LAYLHVTQPR.Y |
| | | | | | | (SEQ ID No.: 18) |
| 620.8552 | 1239.6958 | 1239.5367 | 0.1591 | 79 | 2.6e−07 | R.TDEYGGSLENR.C |
| | | | | | | (SEQ ID No.: 19) |
| 965.6361 | 1929.2577 | 1928.9956 | 0.2621 | 65 | 2.6e−06 | R.ELGLQAVAQGDADLVAFGR.H |
| | | | | | | (SEQ ID No.: 20) |

Characterization of Catalytic Properties of Recombinant AaDBR2 using Artemisinic Aldehyde as Substrate

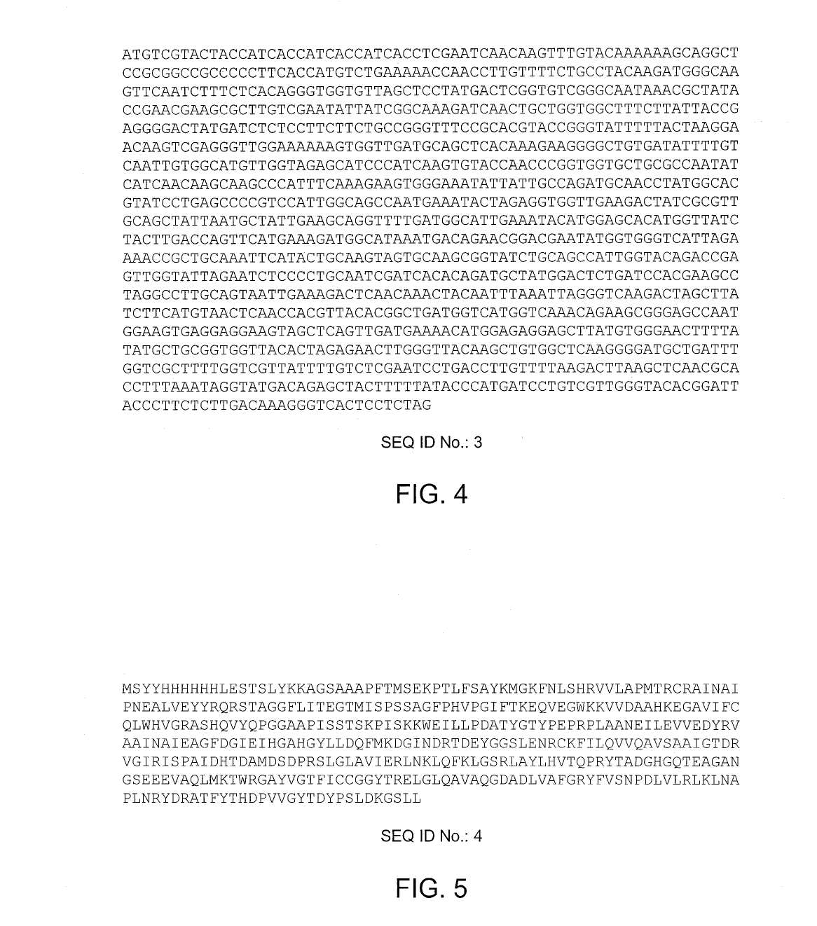

To test the catalytic properties of the gene product, designated AaDBR2, corresponding to GSTSUB_{—}50_F07, its full length cDNA clone (pDEST17-AaDBR2) was obtained by SMART-RACE-PCR. The nucleotide sequence of the open reading frame of the DNA insert of pDEST17-AaDBR2 is given in FIG. 4 (SEQ ID No. 3). This ORF encodes a 415-amino acid protein with a predicted molecular mass of 45.6 kDa. For initial functional studies, plasmid pDEST17-AaDBR2 was introduced into the Rosetta™ 2(DE3) E. coli strain (Novagen). The transgenic Rosetta™ 2(DE3) cells were grown and induced with 1 mM IPTG. The recombinant AaDBR2 protein was purified from cell-free extracts and the corresponding protein product including the N-terminal His tag fusion is given in FIG. 5 (SEQ ID No. 4). The purified AaDBR2 protein was assayed with various substrates followed by analysis by gas chromatography/mass spectrometry. FIGS. 6 (c and d) shows the results of this analysis indicating the NADPH-dependent formation of (11R)-dihydroartemisinic aldehyde as the major product using artemisinic aldehyde as a substrate. Control experiments with boiled enzyme preparation and in the absence of NADPH did not support the production of dihydroartemisinic aldehyde. It is predicted that the wild type product of AaDBR2 will have similar artemisinic aldehyde double bond reductase activity as the His tag fusion protein product of pDEST17-AaDBR2.

Comparison of Catalytic Properties of Recombinant AaDBR2 with other Substrates

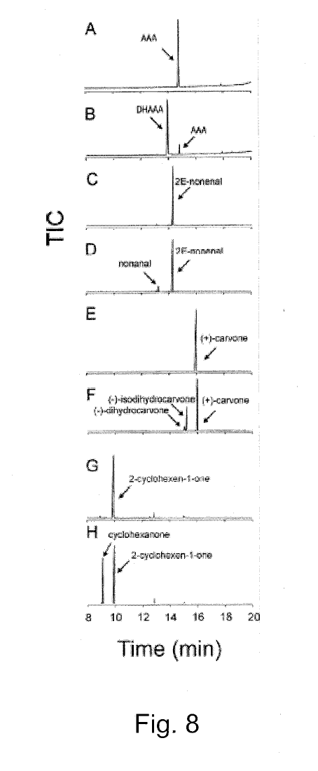

AaDBR2 was also tested with other potential substrates, including arteannuin B, artemisinic acid, artemisinic alcohol, artemisitene, (+)-carvone, coniferyl aldehyde, 2-cyclohexen-1-one, 2E-hexenal, 2E-nonenal, (+)-α-pinene, (+)-pulegone, and sabinone. The results indicate that, in addition to artemisinic aldehyde, AaDBR2 has activity on 2-cyclohexen-1-one, (+)-carvone, and low activity on 2E-nonenal (about 12% of the rate for artemisinic aldehyde; FIG. 8). Based on comparison with a standard 5:1 mixture of (+)-dihydrocarvone/(+)-isodihydrocarvone and assuming that the configuration at C4 was retained, the major and minor products of (+)-carvone were identified as (−)-(1R,4S)-isodihydrocarvone and (−)-(1S,4S)-dihydrocarvone, which were produced in a 5:1 ratio. No activity was detected with arteannuin B, artemisinic acid, artemisinic alcohol, artemisitene, coniferyl aldehyde, 2E-nonenal, (+)-α-pinene, (+)-pulegone, and sabinone. The pH optimum of AaDBR2 was determined to be pH 7.5. At pH 5.5, the enzyme activity was completely inhibited; at pH 9.0, it retained about 25% of its activity at pH 7.5. Preliminary experiments indicated that the enzyme was also active in the presence of NADH.

Kinetic parameters were determined for artemisinic aldehyde, 2-cyclohexen-1-one, (+)-carvone, NADPH, and NADH (Table 2). Relative to 2-cyclohexen-1-one and (+)-carvone, the reductase was very specific for artemisinic aldehyde, for which the Kₘ was more than 30-fold lower (Table 2). The enzyme is also highly specific for. NADPH with a Kₘ of 95 μM, as compared with more than 770 μM for NADH.

TABLE 2

| Substrate | Kₘ (μM) | k_{cat} (s⁻¹) | k_{cat}/Kₘ (mM⁻¹s⁻¹) |
|---|---|---|---|
| artemisinic aldehyde | 19 | 2.6 | 140 |
| 2-cyclohexen-1-one | 790 | 1.8 | 2.3 |
| (+)-carvone | 650 | 0.86 | 1.3 |
| NADPH | 95 | 2.5 | 26 |
| NADH | 770 | 1.3 | 1.7 |

### Use of AaDBR2 in Production of (11-R)-dihydroartemisinic Acid in Yeast

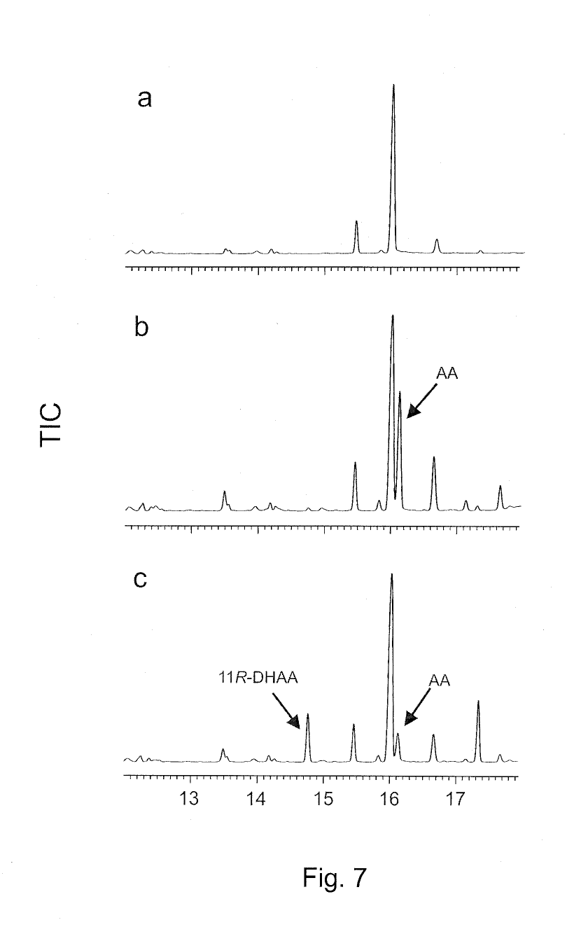

In order to test the effect of expression of AaDBR2 on yeast metabolism, three yeast strains were developed: a control strain containing three empty vectors; a strain which reconstitutes the artemisinin pathway up to artemisinic acid by expressing farnesyl diphosphate synthase (FPS), amorpha-4,11-diene synthase (ADS) and amorpha-4,11-hydroxylase (CYP71AV1); and a strain which additionally expresses AaDBR2. As indicated in FIG. 7a, the control strain does not produce artemisinin-related compounds such as artemisinic acid or dihydroartemisinic acid. Also, as expected the strain expressing CYP71AV1, but not AaDBR2, accumulated artemisinic acid, similar to a previous report (Ro et al., 2006). In this case the level of artemisinic acid in was 29.4 (+/−4.7) μg/mL culture. In the strain expressing AaDBR2, (11R)-dihydroartemisinic acid accumulated to a level of 15.7 (+/−1.4) μg/mL culture (and artemisinic acid accumulated to 11.8 (+/−2.8)). This demonstrates the utility of the AaDBR2 gene in the production of (11R)-dihydroartemisinic acid.

### Homologues of AaDBR2

Table 3 provides mutations of AaDBR2 which are expected to result in proteins that retain artemisinic aldehyde double bond reductase activity. The protein may contain any one of or combination of such mutations. Nucleotide substitutions which either retain the same amino acid sequence as SEQ ID No.: 2 or changes it to one of the mutant amino acid sequences in Table 3 would comprise a functional nucleic acid molecule of the present invention. Such mutations may be created by methods as described in Sambrook et al. 2001 and Ausubel et al. eds. 2001.

TABLE 3

| Amino acid position in | AaDBR2 | |
|---|---|---|
| SEQ ID No.: 2 | (SEQ ID No.: 2) | mutant |
| 42 | V | A |
| 51 | A | P |
| 67 | A | P |
| 116 | G | N |
| 151 | A | G |
| 152 | A | T |
| 153 | N | E |
| 164 | V | Q |
| 167 | I | L |
| 213 | I | L |
| 222 | A | S |
| 248 | R | L |
| 249 | S | Q |
| 304 | K | R |
| 305 | T | S |
| 306 | W | M |
| 325 | L | V |
| 350 | L | M |
| 364 | A | K |
| 369 | H | Q |

References: The disclosures of the following references are incorporated herein by reference in their entirety.
Altschul, S. F., Gish, W., Miller, W., Myers, E. W., & Lipman, D. J. (1990), “Basic local alignment search tool”, Journal of Molecular Biology, vol. 215, pp. 403-410.
Ausubel et al. eds. (2001), Current Protocols in Molecular Biology, (John Wiley & Sons, Somerset, N.J.)
Bagge, M., Xia, X., & Lubberstedt, T. (2007), “Functional markers in wheat”, Curr. Opin. Plant Biol., vol. 10, no. 2, pp. 211-216.
Bertea, C. M., Freije, J. R., van der Woude H., Verstappen, F. W., Perk, L., Marquez, V., de Kraker, J. W., Posthumus, M. A., Jansen, B. J., de Groot, A., Franssen, M. C., & Bouwmeester, H. J. (2005), “Identification of intermediates and enzymes involved in the early steps of artemisinin biosynthesis in Artemisia annua”, Planta Med., vol. 71, no. 1, pp. 40-47.
Bouwmeester, H. J., Wallaart, T. E., Janssen, M. H., van Loo, B., Jansen, B. J., Posthumus, M. A., Schmidt, C. 0., de Kraker, J. W., Konig, W. A., & Franssen, M. C. (1999), “Amorpha-4,11-diene synthase catalyses the first probable step in artemisinin biosynthesis”, Phytochemistry, vol. 52, no. 5, pp. 843-854.
Cahoon, E. B., Carlson, T. J., Ripp, K. G., Schweiger, B. J., Cook, G. A., Hall, S. E., & Kinney, A. J. (1999), “Biosynthetic origin of conjugated double bonds: production of fatty acid components of high-value drying oils in transgenic soybean embryos”, Proceedings of the National Academy of Sciences (U.S.A.), vol. 96, pp. 12935-12940.
Chang, Y. J., Song, S. H., Park, S. H., & Kim, S. U. (2000), “Amorpha-4,11-diene synthase of Artemisia annua: cDNA isolation and bacterial expression of a terpene synthase involved in artemisinin biosynthesis”, Archives of Biochemistry and Biophysics, vol. 383, no. 2, pp. 178-184.
Chang, M. C. Y., Eachus, R. A., Trieu, W., Ro, D.-K. and Keasling, J. D. (2007), “Engineering Escherichia coli for production of functionalized terpenoids using plant P450s”, Nature Chem. Biol., vol. 3, pp. 274-277.
Chou, H.-H. & Holmes, M. H. (2001), “DNA sequence quality trimming and vector removal”, Bioinformatics, vol. 17, pp. 1093-1104.
Comai, L. & Henikoff, S. (2006), “TILLING: practical single-nucleotide mutation discovery”, The Plant Journal, vol. 45, no. 4, pp. 684-694.
Corey, E. J. & Suggs, J. W. (1975), “Pyridinium chlorochromate: An efficient reagent for oxidation of primary and secondary alcohols to carbonyl compounds”, Tetrahedron Letters, vol. 31, pp. 2647-2650.
Duke, M. V., Paul, R. N., Elsohly, H. N., Sturtz, G., & Duke, S. O. (1994), “Localization of artemisinin and artemisitene in foliar tissues of glanded and glandless biotypes of Artemisia annua”, Int. J. Plant Sci., vol. 155, pp. 365-372.
Duke, S. O. & Paul, R. N. (1993), “Development and fine structure of the glandular trichomes of Artemisia annua L.”, Int. J. Plant Sci., vol. 154, pp. 107-118.
Elferaly, F. S. (1990), Method for the isolation of artemisinin from Artemisia annua, U.S. Pat. No. 4,952,603.
Ewing, B., Hillier, L., Wendl, M. C., & Green, P. (1998), “Base-calling of automated sequencer traces using phred I. Accuracy assessment”, Genome Res., vol. 8, pp. 175-185.
Gang, D. R., Wang, J., Dudareva, N., Nam, K. H., Simon, J. E., Lewinsohn, E., & Pichersky, E. (2001), “An investigation of the storage and biosynthesis of phenylpropenes in sweet basil”, Plant Physiol, vol. 125, no. 2, pp. 539-555.
Gupta, S. K., Singh, P., Bajpai, P., Ram, G., Singh, D., Gupta, M. M., Jain, D. C., Khanuja, S. P., & Kumar, S. (2002), “Morphogenetic variation for artemisinin and volatile oil in Artemisia annua”, Ind Crops Products, vol. 16, pp. 217-224.
Henikoff, S., Till, B. J., & Comai, L. (2004), “TILLING. Traditional mutagenesis meets functional genomics”, Plant Physiol, vol. 135, no. 2, pp. 630-636.
Jung, M., Lee, K., & Jung, H. (2001), “First synthesis of (+)-deoxyartemisitene and its novel C-11 derivatives”, Tetrahedron Lett, vol. 42, pp. 3997-4000.
Konieczny, A. & Ausubel, F. M. (1993), “A procedure for mapping Arabidopsis mutations using co-dominant ecotype-specific PCR-based markers”, The Plant Journal, vol. 4, no. 2, pp. 403-410.
Kumar, S. (2002), Method for maximization of artemisinin production by the plant Artemisia annua, U.S. Pat. No. 6,393,763.
Lange, B. M., Wildung, M. R., Stauber, E. J., Sanchez, C., Pouchnik, D., & Croteau, R. (2000), “Probing essential oil biosynthesis and secretion by functional evaluation of expressed sequence tags from mint glandular trichomes”, Proc. Natl. Acad. Sci. U.S.A, vol. 97, no. 6, pp. 2934-2939.
Logeman, J., Schell, J., & Willmitzer, L. (1987), “Improved Method for the Isolation of RNA from Plant Tissues”, Analytical Biochemistry, vol. 163, pp. 16-20.
Martin, V. J., Pitera, D. J., Withers, S. T., Newman, J. D., & Keasling, J. D. (2003), “Engineering a mevalonate pathway in Escherichia coli for production of terpenoids”, Nat. Biotechnol., vol. 21, no. 7, pp. 796-802.
Miller, R. T., Christoffels, A. G., Gopalakrishnan, C., Burke, J., Ptitsyn, A. A., Broveak, T. R., & Hide, W. A. (1999), “A comprehensive approach to clustering of expressed human gene sequence: the sequence tag alignment and consensus knowledge base”, Genome Res., vol. 9, no. 11, pp. 1143-1155.
O'Neill, P. M. (2005), “The therapeutic potential of semi-synthetic artemisinin and synthetic endoperoxide antimalarial agents”, Expert Opin. Investig. Drugs, vol. 14, no. 9, pp. 1117-1128.
Pfaff, T. & Kahl, G. (2003), “Mapping of gene-specific markers on the genetic map of chickpea (Cicer arietinum L.)”, Mol. Genet. Genomics, vol. 269, no. 2, pp. 243-251.
Rathore, D., McCutchan, T. F., Sullivan, M., & Kumar, S. (2005), “Antimalarial drugs: current status and new developments”, Expert Opin. Investig. Drugs, vol. 14, no. 7, pp. 871-883.
Ro, D. K., Paradise, E. M., Ouellet, M., Fisher, K. J., Newman, K. L., Ndungu, J. M., Ho, K. A., Eachus, R. A., Ham, T. S., Kirby, J., Chang, M. C., Withers, S. T., Shiba, Y., Sarpong, R., & Keasling, J. D. (2006), “Production of the antimalarial drug precursor artemisinic acid in engineered yeast”, Nature, vol. 440, no. 7086, pp. 940-943.
Robert, A., Coppel, Y., & Meunier, B. (2002), “Alkylation of heme by the antimalarial drug artemisinin”, Chem. Commun. (Camb.) no. 5, pp. 414-415.
Rotheim, P. (2002), Plant-Derived Drugs: Products, Technologies and Applications, Business Communications Co., Norwalk, B-121.
Sambrook, J. et al. (2001), Molecular Cloning: A Laboratory Manual (Third Edition) (Cold Spring Harbor Laboratory Press, Cold Spring Harbor, N.Y.).
Sandal, N., Krusell, L., Radutoiu, S., Olbryt, M., Pedrosa, A., Stracke, S., Sato, S., Kato, T., Tabata, S., Parniske, M., Bachmair, A., Ketelsen, T., & Stougaard, J. (2002), “A genetic linkage map of the model legume Lotus japonicus and strategies for fast mapping of new loci”, Genetics, vol. 161, no. 4, pp. 1673-1683.
Slade, A. J. & Knauf, V. C. (2005), “TILLING moves beyond functional genomics into crop improvement”, Transgenic Res., vol. 14, no. 2, pp. 109-115.
Stintzi, A. & Browse, J. (2000) GenBank Accession No. AAG15379, Sep. 21, 2000.
Stone, R. T., Grosse, W. M., Casas, E., Smith, T. P., Keele, J. W., & Bennett, G. L. (2002), “Use of bovine EST data and human genomic sequences to map 100 gene-specific bovine markers”, Mammalian Genome, vol. 13, no. 4, pp. 211-215.
Strassner, J., Schaller, F., Frick, U. B., Howe, G. A., Weiler, E. W., Amrhein, N., Macheroux, P. & Schaller, A. (2001) GenBank Accession No. Q9FEW9, Mar. 21, 2001.
Sy, L. K. & Brown, G. D. (2002), “The role of the 12-carboxylic acid group in the spontaneous autoxidation of dihydroartemisinic acid”, Tetrahedron, vol. 58, pp. 909-923.
Teoh, K. H., Polichuk, D. R., Reed, D. W., Nowak, G., & Covello, P. S. (2006), “Artemisia annua L. (Asteraceae) trichome-specific cDNAs reveal CYP71AV1, a cytochrome P450 with a key role in the biosynthesis of the antimalarial sesquiterpene lactone artemisinin”, FEBS Letters, vol. 580, no. 5, pp. 1411-1416.
Teoh, K. H., Reed, D. W., Polichuk, D. R., & Covello, P. S. (2007), World Patent publication WO 2007/112596.
Torrell, M., Garcia-Jacas, N., Susanna, A., & Valles, J. (1999), “Phylogeny in Artemisia (Asteraceae, Anthemideae) inferred from nuclear ribosomal DNA (ITS) sequences”, Taxon, vol. 48, p. 721.
Trotter, E. W., Collinson, E. J., Dawes, I. W., & Grant, C. M. (2006), “Old yellow enzymes protect against acrolein toxicity in the yeast Saccharomyces cerevisiae”, Applied and Environmental Microbiology, vol. 72, no. 7, pp. 4885-4892.
van Agtmael, M. A., Eggelte, T. A., & van Boxtel, C. J. (1999), “Artemisinin drugs in the treatment of malaria: from medicinal herb to registered medication”, Trends Pharmacol. Sci., vol. 20, no. 5, pp. 199-205.
van de Loo, F. J., Turner, S., & Somerville, C. (1995), “Expressed sequence tags from developing castor seeds”, Plant Physiology, vol. 108, pp. 1141-1150.
von Rudloff, E. (1963), Can J. Chem. vol. 41, pp. 2876-2881.
Wallaart, T. E., Bouwmeester, H. J., Hille, J., Poppinga, L., & Maijers, N. C. (2001), “Amorpha-4,11-diene synthase: cloning and functional expression of a key enzyme in the biosynthetic pathway of the novel antimalarial drug artemisinin”, Planta, vol. 212, no. 3, pp. 460-465.
Wallaart, T. E., van Uden, W., Lubberink, H. G., Woerdenbag, H. J., Pras, N., & Quax, W. J. 1999, “Isolation and identification of dihydroartemisinic acid from Artemisia annua and its possible role in the biosynthesis of artemisinin”, J. Nat. Prod., vol. 62, no. 3, pp. 430-433.
Watson, L. E., Evans, T. M., & Boluarte, T. (2000), “Molecular phylogeny and biogeography of tribe Anthemideae (Asteraceae), based on chloroplast ndhF”, Mol. Phylogen. Evol., vol. 15, pp. 59-69.
Wilairatana, P., Krudsood, S., Treeprasertsuk, S., Chalermrut, K., & Looareesuwan, S. (2002), “The future outlook of antimalarial drugs and recent work on the treatment of malaria”, Arch. Med. Res., vol. 33, no. 4, pp. 416-421.
Wu, Y. (2002), “How might qinghaosu (artemisinin) and related compounds kill the intraerythrocytic malaria parasite? A chemist's view”, Acc. Chem. Res., vol. 35, no. 5, pp. 255-259.
Yadav, J. S., Babu, R. S., & Sabitha, G. (2003), “Stereoselective total synthesis of (+)-artemisinin”, Tetrahedron Lett, vol. 44, pp. 387-389.

Other advantages that are inherent to the structure are obvious to one skilled in the art. The embodiments are described herein illustratively and are not meant to limit the scope of the invention as claimed. Variations of the foregoing embodiments will be evident to a person of ordinary skill and are intended by the inventor to be encompassed by the following claims.

1. Isolated nucleic acid molecule comprising a nucleotide sequence having at least 70% nucleotide sequence identity to the nucleotide sequence as set forth in nucleotides 63 to 1226 of SEQ ID No: 1, and encoding an artemisinic aldehyde double bond reductase.

2. Isolated nucleic acid molecule comprising a nucleotide sequence encoding an artemisinic aldehyde double bond reductase having an amino acid sequence with at least 70% amino acid sequence identity to SEQ ID No. 2.

3. The nucleic acid molecule according to claim 2, wherein the amino acid sequence identity is at least 80%.

4. The nucleic acid molecule according to claim 2, wherein the amino acid sequence identity is at least 90%.

5. The nucleic acid molecule according to claim 2, wherein the amino acid sequence identity is at least 95%.

6. The isolated nucleic acid molecule according to claim 1 having a nucleotide sequence comprising SEQ ID No.: 1.

7. The isolated nucleic acid molecule of claim 1 derived from Artemisia annua.

8. Purified or partially purified artemisinic aldehyde double bond reductase having an amino acid sequence with at least 70% amino acid sequence identity to SEQ ID No. 2.

9. The reductase according to claim 8, wherein the amino acid sequence identity is at least 80%.

10. The reductase according to claim 8, wherein the amino acid sequence identity is at least 90%.

11. The reductase according to claim 8, wherein the amino acid sequence identity is at least 95%.

12. The reductase according to claim 8 having an amino acid sequence comprising SEQ ID No.: 2.

13. Purified or partially purified artemisinic aldehyde double bond reductase encoded by the isolated nucleic acid molecule as defined in claim 1.

14-15. (canceled)

16. Process for producing dihydroartemisinic aldehyde, dihydroartemisinic acid and/or artemisinin comprising expressing or overexpressing one or more isolated nucleic acid molecules as defined in claim 1 in a host cell.

17. The process according to claim 16, further comprising expressing or overexpressing in the host cell one or more nucleic acid molecules encoding farnesyl diphosphate synthase, amorpha-4,11-diene synthase, amorpha-4,11-diene hydroxylase, alcohol dehydrogenase, aldehyde dehydrogenase, or any combination thereof.

18. Process for producing dihydroartemisinic aldehyde, dihydroartemisinic acid and/or artemisinic acid comprising producing or overproducing in a host cell one or more artemisinic aldehyde double bond reductases as defined in claim 8.

19. The process according to claim 16, wherein the host cell is a plant cell.

20. The process according to claim 16, wherein the host cell is a yeast cell.

21. The process according to claim 16, wherein the host cell is a bacterial cell.

22. Method of selecting or developing plants with altered dihydroartemisinic aldehyde, dihydroartemisinic acid, artemisinic acid and/or artemisinin levels in a population of plants that naturally produces dihydroartemisinic aldehyde, dihydroartemisinic acid, artemisinic acid and/or artemisinin comprising:
detecting a target plant having altered levels of dihydroartemisinic aldehyde, dihydroartemisinic acid, artemisinic acid and/or artemisinin compared to a control plant provided under similar conditions;
isolating at least a portion of an artemisinic aldehyde double bond reductase gene of the target plant and comparing the nucleotide sequence of said at least a portion to SEQ ID No.: 2 to detect a variation from SEQ ID No. 2;
detecting the variation in other plants;
selectively breeding the plants with the variation to produce a population of plants having altered levels of dihydroartemisinic aldehyde, dihydroartemisinic acid, artemisinic acid and/or artemisinin compared to a population of control plants produced under similar conditions.

23. Method of increasing dihydroartemisinic aldehyde, dihydroartemisinic acid, artemisinic acid and/or artemisinin levels in a population of plants that naturally produces dihydroartemisinic aldehyde, dihydroartemisinic acid, artemisinic acid and/or artemisinin comprising:
providing a population of mutated plants in which at least a portion of the mutated plants have an altered expression of an artemisinic aldehyde double bond reductase gene or altered activity of an artemisinic aldehyde double bond reductase enzyme compared to a control plant provided under similar conditions;
detecting a target mutated plant within the population of mutated plants, the target mutated plant having an altered expression of an artemisinic aldehyde double bond reductase gene or altered activity of an artemisinic aldehyde double bond reductase enzyme compared to a control plant provided under similar conditions,
said detecting comprising using primers developed from a nucleic acid molecule as defined in any one of claims 1 to 6 to PCR amplify regions of the artemisinic aldehyde double bond reductase gene from mutated plants in the population of mutated plants, identifying mismatches between the amplified regions and corresponding regions in wild-type gene of the plant that naturally produces dihydroartemisinic aldehyde, dihydroartemisinic acid, artemisinic acid and/or artemisinin that lead to the altered expression or altered activity, and identifying the mutated plant that contains the mismatches; and,
selectively breeding the target mutated plant to produce a population of plants having altered expression of artemisinic aldehyde double bond reductase gene or altered activity of artemisinic aldehyde double bond reductase enzyme compared to a population of control plants produced under similar conditions.
